(19) United States
(12) Patent Application Publication
(10) Pub. No.: US 2016/0206372 A1
(43) Pub. Date: Jul. 21, 2016

(54) METHOD OF NERVE ABLATION AND USES THEREOF

(71) Applicant: Daniel Rivlin, Miami Beach, FL (US)

(72) Inventor: Daniel Rivlin, Miami Beach, FL (US)

(21) Appl. No.: 14/996,612

(22) Filed: Jan. 15, 2016

Related U.S. Application Data
(60) Provisional application No. 62/103,917, filed on Jan. 15, 2015.

(51) Int. Cl. A61B 18/14; A61M 19/00

(52) U.S. Cl. CPC A61B 18/1492; A61M 19/00; A61B 2018/00642

(57) ABSTRACT

Procedures for ablating nerves, in particular, those innervating the platysma muscle are provided. The ablation of nerves in the platysma is utilized in medical treatments and for cosmetic interventions.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 62/103,917, entitled “METHOD OF NERVE ABLATION AND USES THEREOF”, filed Jan. 15, 2015. The content of the above referenced application is incorporated herein by reference in its entirety.

### FIELD OF THE INVENTION

Embodiments are directed to a method of nerve ablation for use in medical and cosmetic procedures. In particular, targeted ablation sites are the nerves that innervate the platysma muscle.

### BACKGROUND

Results using Botox in face lifting procedures were reported approximately ten years ago. Botox was injected into the platysma muscle which arises from the collarbone, proceeds upwards in a slanting manner along the sides of the neck and drops at the lower lip and corner of the mouth sideways and down. The platysma muscle has no necessary action and is probably an evolutionary remnant. As people age, this muscle tends to contract and form bands within it. A vital part of the facelift procedure is to release these bands forcefully by pulling and suturing. However, Botox is not a viable alternative to face lifting for the following reasons: 1) the number of units necessary to achieve complete relaxation of the platysma muscle border on toxic concentrations and 2) cost of that many units is prohibitive, especially since the results are effective for about three months.

### SUMMARY

Embodiments of the invention are directed, in part, to methods of performing nerve ablation comprising: mapping one or more ablation sites; administering to a subject concentrated anesthetic in low volumes at or near an ablation target site: targeting a nerve or nerve branch at one or more ablation target sites; advancing an ablation element to the ablation target site; activating the ablation element at least once to ablate the nerve.

In an embodiment, the method is performed for cosmetic reasons, such, as for example, a facelift. In other embodiments, the method is performed for treatment or management of symptoms of a neurological disease or disorder, e.g. muscle twitching, dystonia etc.

In an embodiment, a target ablation site is the platysma of a subject.

Other aspects are described infra.

### BRIEF DESCRIPTION OF THE DRAWINGS

The patent or application contains at least one drawing executed in color. Copies of this patent or patent application publication with color drawings will be provided by the Office upon request and payment of the necessary fee.

FIG. 1 is a photograph of a frontal view showing potential ablation sites in the neck of a subject;

FIG. 2 is a photograph of a side view showing potential ablation sites in the neck of a subject;

FIG. 3 is a photograph of a frontal view of a point of entry of a catheter entering under the skin and the ablation site from that point of entry;

FIG. 4 is the same as FIG. 3 except it also shows cervical nerve distribution in relation to the ablation sites;

FIG. 5 is a photograph showing ablation sites numbered according to order of stacked ablations. Ablations are preferably carried out beginning from the inferior end first (e.g. 1, 2, etc.);

FIG. 6A is a photograph showing cervical nerve distribution in the neck and lower face;

FIG. 6B is an overlay of FIG. 6A depicting areas safe for nerve ablation; and

FIG. 7 is a photograph showing cervical nerve distribution in relation to ablation sites.

### DETAILED DESCRIPTION

Embodiments of the invention are directed to ablation of nerves for use in medical or cosmetic procedures.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As defined herein, an “effective” amount of a compound or agent (i.e., an effective dosage) means an amount sufficient to produce a (e.g., clinically) desirable result.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Procedures for Ablating Nerves

Current treatments use radiofrequency devices as an alternative to Botox. Radiofrequency devices generally consist of a catheter attached to a radio frequency generator, and within the catheter is an internal nerve stimulator. In general, the technique to ablate nerves consists of first mapping out the nerves with an external nerve stimulator. Then, the catheter is inserted in the mapped out areas and the nerves are specifically located with the internal nerve stimulator. Once the nerves are located, they are then ablated with radiofrequency. In general, when radiofrequency devices are used to ablate nerves, nerve blocks are administered prior to the nerve ablation.

The inventor discovered that the nerve blocks administered to patients during such procedures prevented the accurate location of nerves with the internal nerve stimulator. Use of such techniques without anesthesia made it extremely effective and long-lasting; however, this renders such procedures extremely painful and therefore nonmarketable.

The Platysma Muscle: The platysma muscle belongs, as all other facial muscles, to the group of cross-striated musculature. The platysma muscle is a broad sheet of muscle arising from the pectoral (chest) and deltoid (shoulder) muscles and rises over the collarbone (clavicle), proceeding upward in a slanting manner along the sides of the neck. The muscle draws the lower lip and corner of the mouth sideways and down, partially opening the mouth, as in an expression of surprise or fright. When all the fibers work to their maximum, the platysma increases the diameter of the neck as well, as may be seen during the intensive breathing of a sprinting runner.

The platysma is counted among the mimic musculature, is a superficially proceeding muscle and—unlike other muscles—is directly connected to the skin. Due to the direct insertion into the skin, it can change the facial expression along with other facial muscles. Since the platysma is intensely innervated —similar to the tongue and eye muscles, damage to the platysma may lead to dysfunction of the facial expression in terms of paralysis.

The methods embodied herein are painless and prevent damage or injury, as the locations of the nerves are identified and the ablation sites are mapped prior to ablation ensuring accurate nerve ablation in a subject.

Target Ablation Sites: In an embodiment, a method of performing a nerve ablation procedure comprises: mapping one or more ablation sites; administering to a subject an effective amount of a concentrated anesthetic in low volumes at or near an ablation target site; targeting a nerve or branches of a nerve at one or more ablation target sites; advancing an ablation element to the ablation target site; and activating the ablation element at least once to ablate the nerve.

In order to map the one or more ablation sites which are to be targeted in a patient, the patient is requested to thrust their jaw out and smile. This maneuver highlights the platysma small bands. The lateral and medial margins of the bands can be marked with a marking pen. The doctor or medical personnel can then take an external nerve stimulator and apply it along the lateral and medial borders and find the “hotspot”. With the internal nerve stimulator, the same can be done; however, one must make sure that the stimulator is underneath the muscle. This can be done by inserting the stimulator when the patient is flexing the platysmal bands. The ablation of the nerve is stacked, wherein a nerve or branches of a nerve are ablated at a first target ablation site, followed by ablating one or more nerve or nerve branches at another target ablation site located superior to the first target ablation site. The target ablation sites comprise one or more nerves or branches of nerves comprising: nerves innervating the platysma, angular nerves, temporal nerves, the cervical nerve (VII) or branches of the cervical nerve (VII).

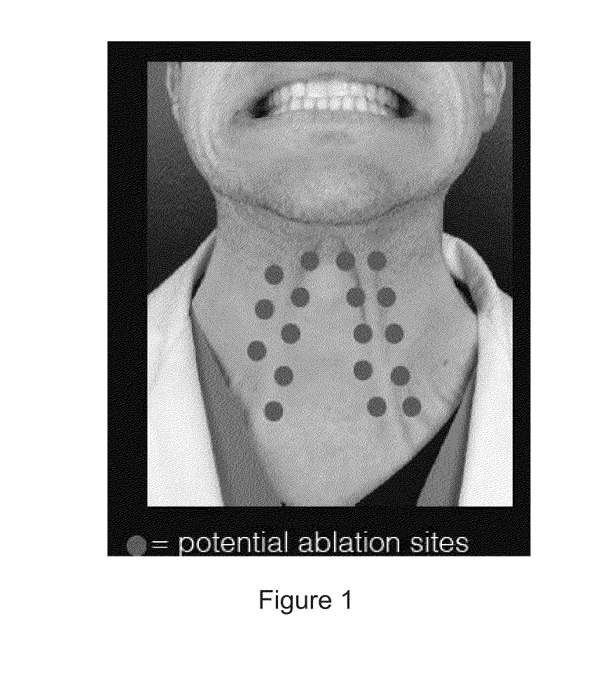

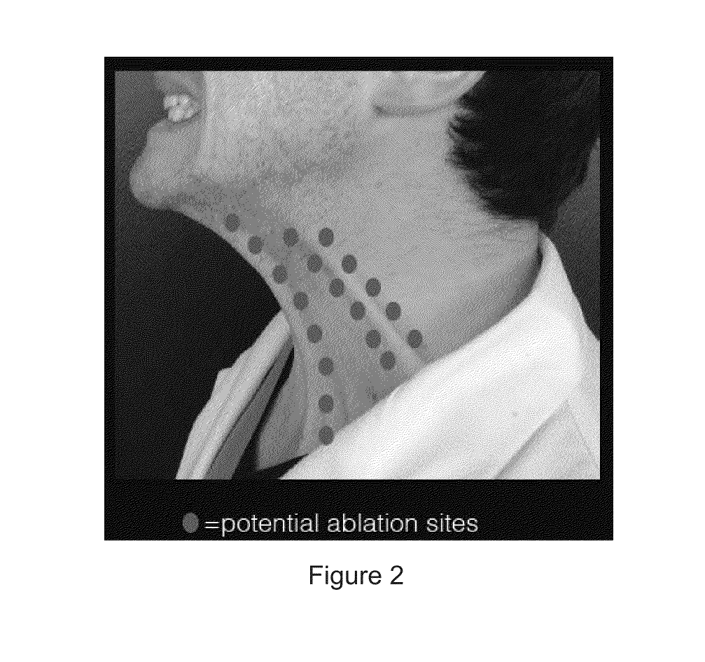

In particular embodiments, the target nerve ablation sites are located in the subject's neck region (FIGS. 1, 2). FIG. 1 is a frontal view of a subject's neck showing potential ablation sites in the neck of a subject, and FIG. 2 is a side view.

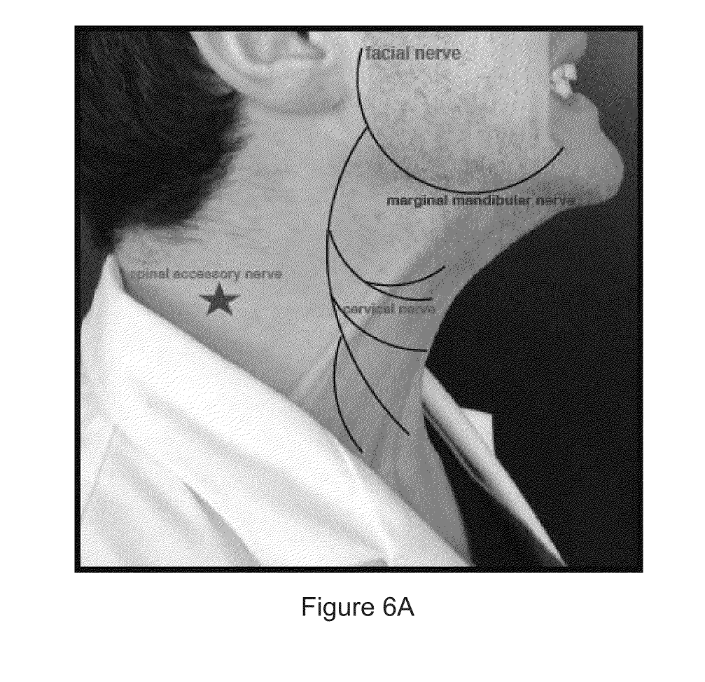

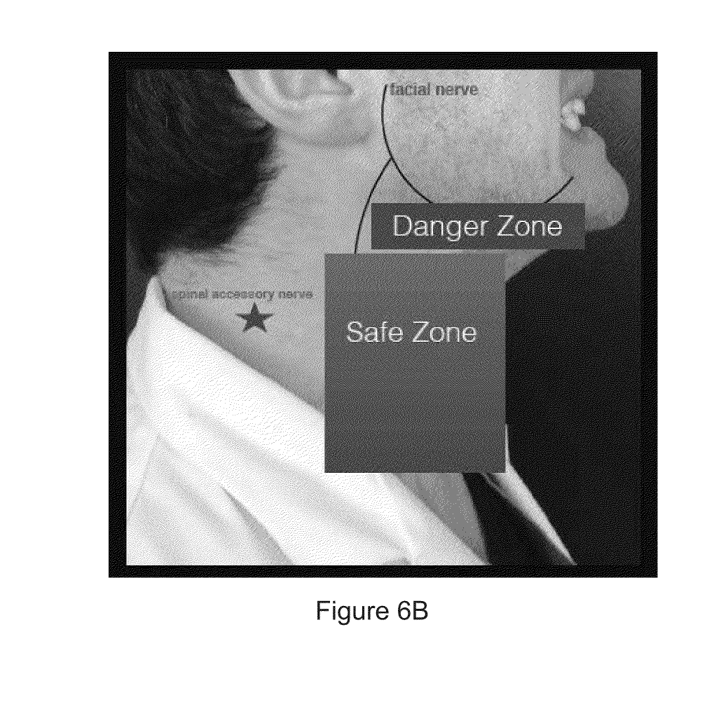

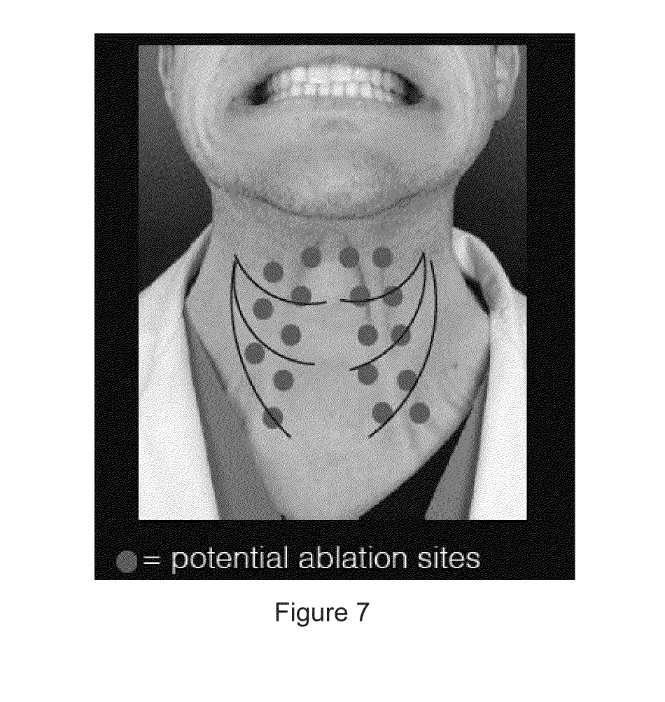

In an embodiment, a target ablation site comprises the nerves that innervate the platysma. In general, the nerves to platysma are (i) the efferent: cervical branch of facial nerve (VII); and, (ii) the transverse cervical nerve (C2, C3)-proprioceptive fibers. FIGS. 6A and 6B depict the cervical nerve distribution in the neck and lower face (FIG. 6A) and areas safe for nerve ablation (FIG. 6B), so as not to injure or damage other nerves and tissues of a subject. FIG. 7 depicts cervical nerve distribution in relation to ablation sites.

In some embodiments, a target ablation site in the platysma includes the cervical nerve (VII) or branches of the cervical nerve (VII). In an embodiment, a target ablation site is one or more branches of the cervical nerve (VII). Care should be taken to avoid the marginal branch of the mandibular nerve as well as the spinal accessory nerve.

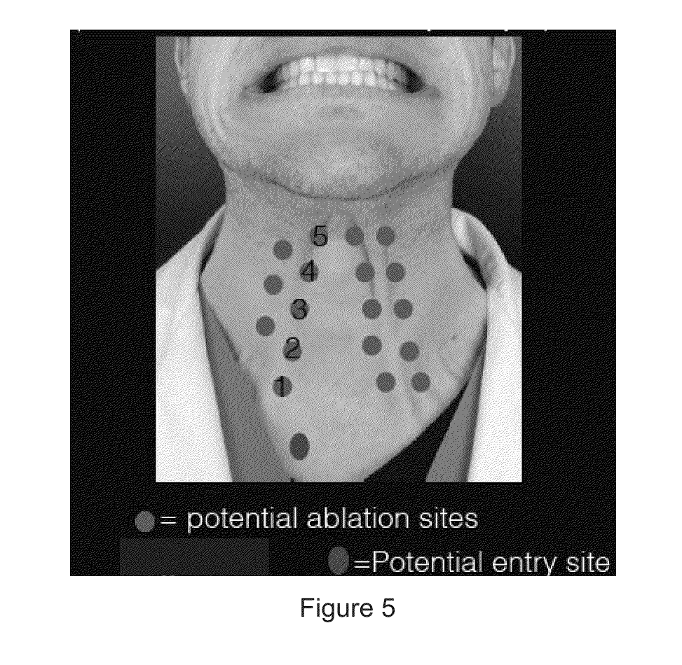

Anesthetics and Pain Management: The method for the effective ablation is conducted in a virtual pain-free manner. The method comprises one or more steps to achieve long-term effective and minimally painful results, the one or more steps comprising: (i) using very concentrated anesthesia in extremely low volumes through the port; (ii) allowing about two to ten minutes for anesthesia to take affect before ablating; (iii) the ablations are stacked so that the nerve is destroyed (FIG. 5). An effective amount of anesthetic is administered topically, subcutaneously, intra-muscularly and/or intravenously.

A drug may be classified by the chemical type of the active ingredient or by the way it is used to treat a particular condition. Each drug can be classified into one or more drug classes. Accordingly, different classes and types of anesthetics can be administered. These include local, topical, or general (inhalational, intra-venous). Only preservative-free local anesthetic agents may be injected intrathecally.

In one embodiment, one or more anesthetics are selected from a local class of anesthetics. In one embodiment, one or more anesthetics are selected from a topical class of anesthetics. In one embodiment, one or more anesthetics are selected from a general class of anesthetics. In one embodiment, a combination of two or more anesthetics is selected from a local, topical, or general class of anesthetics.

Local anesthetic: Local anesthetics are agents that prevent transmission of nerve impulses without causing unconsciousness. They act by binding to fast sodium channels from within (in an open state). Local anesthetics can be either ester- or amide-based. Examples are: Procaine, amethocaine, cocaine, lidocaine (also known as Lignocaine), prilocaine, bupivacaine, levobupivacaine, ropivacaine, mepivacaine, dibucaine.

Ester local anesthetics (e.g., procaine, amethocaine, cocaine, benzocaine, tetracaine) are generally unstable in solution and fast-acting, and allergic reactions are common.

Amide local anesthetics (e.g., lidocaine, prilocaine, bupivicaine, levobupivacaine, ropivacaine, mepivacaine, dibucaine and etidocaine) are generally heat-stable, with a long shelf life (around 2 years). They have a slower onset and longer half-life than ester anesthetics, and are usually racemic mixtures, with the exception of levobupivacaine (which is S(−)-bupivacaine) and ropivacaine (S(−)-ropivacaine). These agents are generally used within regional and epidural or spinal techniques, due to their longer duration of action, which provides adequate analgesia for surgery, labor, and symptomatic relief.

Topical anesthetics: Topical anesthetics are agents that reduce the sensation in the area they are applied on such as the skin, throat, in the nose, front of the eyes, etc. They are available as creams, ointments, solutions, gels or sprays. Topical anesthetics are used as pain relief. They are applied to the skin to treat pain caused by sunburn or other minor burns, insect bites, minor cuts, etc. It is also used to numb the skin before inserting a needle. Examples of topical anesthetics include: benzalkonium chloride/lidocaine, lidocaine, hydrocortisone/lidocaine, phenol, lidocaine/prilocaine, lidocaine/tetracaine, lidocaine/prilocaine, capsaicin/lidocaine/menthol, calamine/pramoxine, methyl salicylate.

Examples of commercially available topical anesthetics include: ORAQIX (generic name: lidocaine/prilocaine), RETRE-GEL (generic name: benzocaine), SARNA SENSITIVE (generic name: pramoxine), VIRACTIN (generic name: tetracaine), NUPERCAINAL (generic name: dibucaine), EMLA (generic name: lidocaine/prilocaine), pramoxine, RECTAGEL HC (generic name: hydrocortisone/lidocaine), Menthocin Patch with Lidocaine (generic name: capsaicin/lidocaine/menthol/methyl salicylate).

General anesthetics: General anesthetics are drugs that act in the CNS. They produce reversible loss of consciousness, thereby causing a generalized loss of sensation. The general anesthetic effect involves the following components:—loss of arousability in response to noxious stimuli;—loss of pain sensation (analgesia) in response to noxious stimuli;—loss of mobility (immobility) in response to noxious stimuli;—loss of memory (amnesia);—attenuation of autonomic responses to noxious stimuli. Some of the general anesthetics are volatile compounds that are given by inhalation. Others are non-volatile compounds that are dissolved and injected intravenously. Examples of general anesthetics include, without limitation: KETALAR (generic name: ketamine), DIPRIVAN (generic name: propofol), PENTOTHAL (generic name: thiopental); INAPSINE (generic name: droperidol); AMIDATE (generic name: etomidate), Brevital Sodium (generic name: methohexital), FORANE (generic name: isoflurane); LUSEDRA (generic name: fospropofol), Propoven (generic name: propofol), Sojourn (generic name: sevoflurane), SUPRANE (generic name: desflurane), ULTANE (generic name: sevoflurane).

Intravenous agents (non-opioid): While there are many drugs that can be used intravenously to produce anesthesia or sedation, the most common are: Barbiturates, Amobarbital (trade name: AMYTAL), Methohexital (trade name: BREVITAL), Thiamylal (trade name: SURITAL), Thiopental (trade name: PENTHOTHAI), Benzodiazepines, Diazepam, Lorazepam, Midazolam, Etomidate, Ketamine, Propofol.

Intravenous opioid analgesic agents: Opioids are rarely used to induce anesthesia, they are frequently used along with other agents such as intravenous non-opioid anesthetics or inhalational anesthetics. Furthermore, they are used to relieve pain of patients before, during, or after surgery. The following opioids have short onset and duration of action and are frequently used during general anesthesia: Alfentanil, Fentanyl, Remifentanil, Sufentanil.

The following agents have longer onset and duration of action and are frequently used for post-operative pain relief: Buprenorphine, Butorphanol, diacetyl morphine, (Diamorphine), Hydromorphone, Levorphanol, Meperidine, Methadone, Morphine, Nalbuphine, Oxycodone, Oxymorphone, Pentazocine.

Inhalational anaesthetics: These include, without limitation: Desflurane, Enflurane, Halothane, Isoflurane, Methoxyflurane, Nitrous oxide, Sevoflurane.

Accordingly, in embodiments, an effective amount of concentrated anesthetic for use in anesthetizing the nerve comprises from about 0.001% volume/volume (v/v) to about 10% v/v of an anesthetic. In another embodiment, an effective amount of concentrated anesthetic for use in anesthetizing the nerve comprises from about 0.001% volume/volume (v/v) to about 10% v/v of one or more anesthetics selected from a local class of anesthetics. In another embodiment, an effective amount of concentrated anesthetic for use in anesthetizing the nerve comprises from about 0.001% volume/volume (v/v) to about 10% v/v of one or more anesthetics selected from a topical class of anesthetics In another embodiment, an effective amount of concentrated anesthetic for use in anesthetizing the nerve comprises from about 0.001% volume/volume (v/v) to about 10% v/v of one or more anesthetics selected from a general class of anesthetics. In one embodiment, an effective amount of concentrated anesthetic for use in anesthetizing the nerve comprises from about 0.001% volume/volume (v/v) to about 10% v/v of a combination of two or more anesthetics selected from a local, topical, or general class of anesthetics.

In an embodiment, an anesthetic for use in performing nerve ablation in a subject comprises mapping one or more ablation sites; administering to a subject an effective amount of concentrated anesthetic in low volumes at or near an ablation target site; targeting a nerve or nerve branch at one or more ablation target sites; advancing an ablation element to the ablation target site; activating the ablation element at least once to ablate the nerve.

In another embodiment, an anesthetic for use in ablating the platysma in a subject comprising: administering to a subject an effective amount of concentrated anesthetic in low volumes at or near an ablation target site in the platysma; targeting a nerve or nerve ending at one or more ablation target sites; advancing an ablation element to the ablation target site; activating the ablation element at least once to ablate the nerve.

In an embodiment, a concentrated anesthetic for use in anesthetizing the nerve comprises from about 0.001% volume/volume (v/v) to about 10% v/v of lidocaine. In another embodiment, a concentrated anesthetic for use in anesthetizing the nerve comprises from about 2% volume/volume (v/v) to about 5% v/v of lidocaine. In another embodiment, a concentrated anesthetic for use in anesthetizing the nerve comprises about 4% v/v of lidocaine.

In an embodiment, a concentrated anesthetic for use in anesthetizing the nerve comprises from about 0.001% volume/volume (v/v) to about 5% v/v of septicaine. In another embodiment, a concentrated anesthetic for use in anesthetizing the nerve comprises from about 0.1% volume/volume (v/v) to about 2% v/v of septicaine. In another embodiment, a concentrated anesthetic for use in anesthetizing the nerve comprises about 1% v/v of septicaine.

In another embodiment, the volume of anesthesia per unit dose comprises from about 0.001 ml to about 10 ml per unit dose. In another embodiment, the volume of anesthesia per unit dose comprises from about 0.01 ml to about 5 ml per unit dose. In another embodiment, the volume of anesthesia per unit dose comprises from about 0.02 ml to about 1 ml per unit dose. In another embodiment, the volume of anesthesia per unit dose comprises from about 0.05 ml to about 0.2 ml per unit dose.

In another embodiment, the time or duration for allowing the anesthesia to anesthetize the nerve prior to nerve ablation comprises from about 30 seconds to about 5 minutes. In another embodiment, the time for allowing the anesthesia to anesthetize the nerve prior to nerve ablation comprises from about 1 minute to about 4 minutes. In another embodiment, the time for allowing the anesthesia to anesthetize the nerve prior to nerve ablation comprises about 2 minutes.

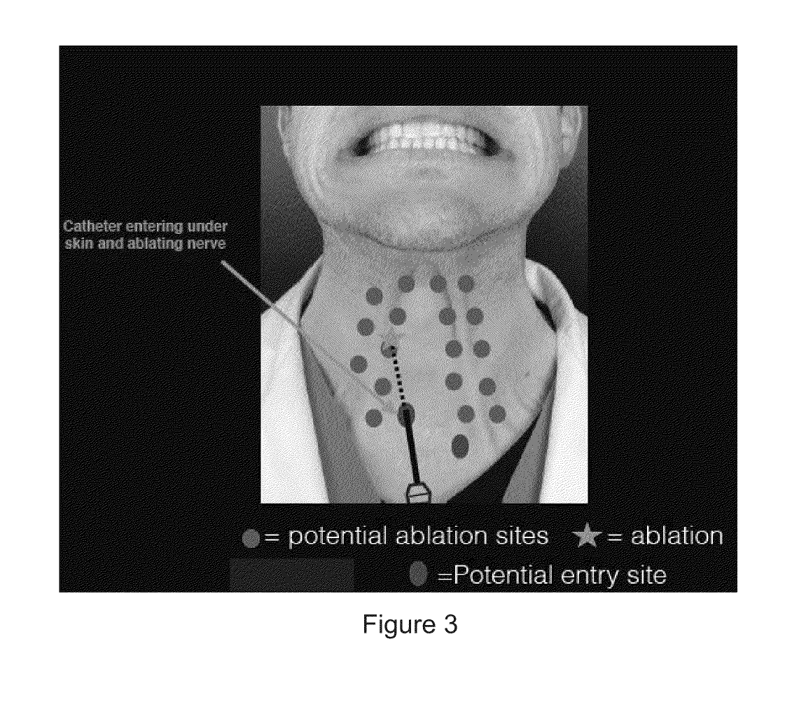

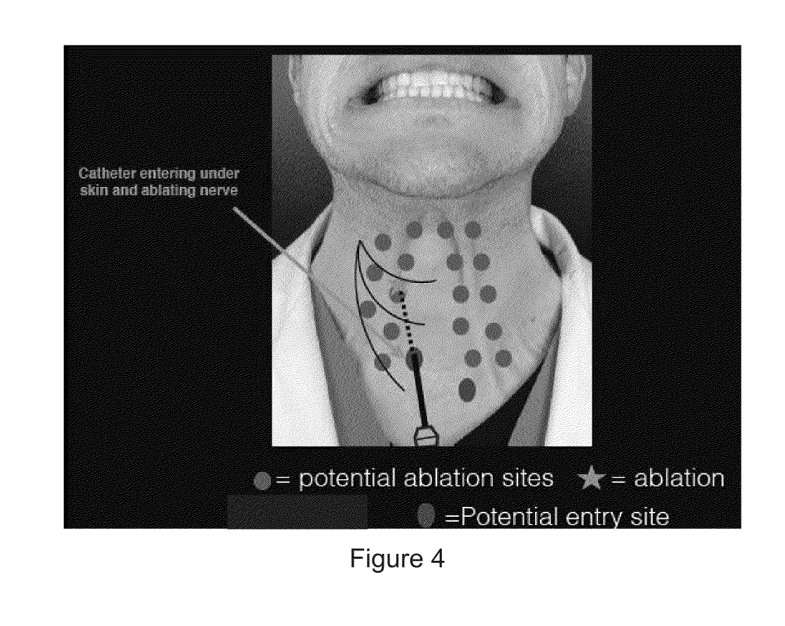

Nerve Ablation: Embodiments of the disclosure are further directed to methods for multi-site RF ablation of cervical nerves for cosmetic changes and other treatments. According to various embodiments, an ablation device may include an elongated shaft having multiple expandable splines near the distal end. The expandable splines may move between a first delivery position and a second expanded position. An RF electrode is mounted on the distal tip of the elongate shaft. The multiple expandable splines comprise expandable curved, arcuate, or other structures to place the RF electrodes at a desired target location. FIG. 3 shows a frontal view of a point of entry of a catheter which enters under the skin. Nerve ablation or the ablation site from that point of entry is indicated. FIG. 4 shows cervical nerve distribution in relation to the ablation sites. Ablations are preferably carried out beginning from the inferior end first, e.g. 1, 2, etc. (FIG. 5).

In an embodiment, the nerve ablations are stacked. Stacking refers to the concept of ablating the entire nerve. For example, if a 1 cm segment of a nerve is ablated, the catheter is then moved exactly 1 cm to ablate the next segment completely. This is done until the entire nerve is treated. The number of ablations necessary varies from nerve to nerve. To ensure a long-term result, almost the entire length of the nerve is ablated. Since the catheter tips can be either 5 mm or 10 mm, as many ablations are conducted as needed to cover the entire nerve depending on the length of the nerve. For example, if the nerve is 3 cm, a 1 cm tip is used and three ablations are conducted.

In embodiments, the ablation element or device delivers one or more intensities of ablation energy per unit of time. The ablation energy comprises: radiofrequency (RF), ultrasound, high intensity ultrasound, high intensity focused ultrasound, thermal energy, microwave, laser, cryogenic energy, mechanical or combinations thereof. In order to confirm that the nerve is ablated, immediately after the ablation the internal nerve stimulator is again turned on at a much higher amperage, for example, about 1.0 millivolts (MV) or about 1.2 MV.

The ablation of the nerve can be carried out using any type of energy, including, without limitation, radiofrequency (RF) energy, ultrasound, high intensity ultrasound, high intensity focused ultrasound, thermal energy, microwave, laser, cryogenic energy, or any type of mechanical means. In an embodiment, the target temperature for ablating a nerve is between about 60° C. to about 100° C. for about 30 seconds to about 5 minutes. In an embodiment, the target temperature for ablating a nerve is about 85° C. for about 30 seconds to about 2 minutes. In an embodiment, the target temperature for ablating a nerve is about 85° C. for about 60 seconds.

In an embodiment, the nerve ablation results in a level five injury to the nerve. Any type of nerve ablation device can be used. In some embodiments, the nerve ablation device is a radiofrequency (RF) device comprising an anesthesia port within the catheter. In embodiments where radiofrequencies are used to ablate the nerves, high frequencies are employed. For example, the cyclical waveform can be applied at a frequency in the range of between 100 Hz and 50,000 Hz.

Uses: Nerve ablations may be required or advised for certain therapeutic and cosmetic procedures.

In an embodiment, a method for performing a cosmetic procedure (e.g. facelift) comprises targeting nerves or branches of nerves innervating the platysma muscle. To effectively ablate the platysma, the anatomy of the platysma needs to be understood, which also includes understanding the innervations as well as surrounding structures that could be destroyed by radiofrequency ablation. The platysma is: innervated by the cervical branch of the facial nerves; it emerges in the posterior auricular triangle and courses anteriorly, which is perpendicular to the long axis of the platysma (see, FIG. 4); it ramifies as it courses anteriorly; the nerve is under the muscle and the branches that cause the banding are medial to the band.

The technique is expanded to ablate the angular and temporal nerves in the glabellar frown complex, and also on the medial borders of the platysmal bands. Either an inferior to superior insertion could be used, or a lateral medial insertion. However, the latter would include multiple insertion points.

In some embodiments, nerve ablations at target sites in the platysma are conducted for medical treatments (e.g. dystonia, muscle twitching, neuralgia, migraine headaches, headaches and the like) or cosmetic reasons due to aging, certain disorders, etc.

Various embodiments disclosed herein are generally described in the context of ablation of the cervical nerve (VII) or branches of the cervical nerve (VII) for medical or cosmetic reasons. It is understood, however, that embodiments of the disclosure have applicability in other contexts, such as performing ablation from within other vessels of the body, including other arteries, veins, and vasculature and other tissues of the body, including various organs.

It is to be understood that even though numerous characteristics of various embodiments have been set forth in the foregoing description, together with details of the structure and function of various embodiments, this detailed description is illustrative only and changes may be made in detail, especially in matters of structure and arrangements of parts illustrated by the various embodiments to the full extent indicated by the broad general meaning of the terms in which the appended claims are expressed.

What is claimed:

1. A method of performing nerve ablation in a subject, comprising: mapping one or more ablation sites; administering to a subject concentrated anesthetic in low volumes at or near an ablation target site; targeting a nerve or nerve branch at one or more ablation target sites; advancing an ablation element to the ablation target site: activating the ablation element at least once to ablate the nerve.

2. The method of claim 1, wherein the ablation element delivers one or more intensities of ablation energy per unit of time to achieve a temperature of between about 70° C. to about 90° C. for at least about 0.5 minutes to about 3 minutes.

3. The method of claim 2, wherein the ablation energy comprises: radiofrequency (RF), ultrasound, high intensity ultrasound, high intensity focused ultrasound, thermal energy, microwave, laser, cryogenic energy, mechanical or combinations thereof.

4. The method of claim 1, wherein the ablation of the nerve is stacked, comprising ablating a nerve or nerve branch at a first target ablation site, followed by one or more nerve or nerve branch ablations at target ablation sites located superior to the first target ablation site.

5. The method of claim 1, wherein an anesthetic is administered topically, locally and/or subcutaneously.

6. The method of claim 1, wherein the concentration of anesthetic is about 0.001% volume/volume (v/v) to about 10% v/v.

7. The method of claim 1, wherein the volume of anesthetic administered comprises between about 0.001 ml to about 10 ml.

8. The method of claim 1, wherein the target ablation sites comprise one or more nerves comprising: nerves affecting platysma, angular nerves, temporal nerves, the cervical nerve (VII) or branches of the cervical nerve (VII).

9. A method of ablating the platysma in a subject comprising: administering to a subject concentrated anesthetic in low volumes at or near an ablation target site in the platysma; targeting a nerve or nerve ending at one or more ablation target sites; advancing an ablation element to the ablation target site; activating the ablation element at least once to ablate the nerve.

10. The method of claim 9, wherein the activated ablation element delivers one or more intensities of ablation energy per unit of time to achieve a temperature of between about 70° C. to about 90° C. for at least about 0.5 minutes to about 3 minutes.

11. The method of claim 10, wherein the ablation energy comprises: radiofrequency (RF), ultrasound, high intensity ultrasound, high intensity focused ultrasound, thermal energy, microwave, laser, cryogenic energy, mechanical or combinations thereof.

12. The method of claim 9, wherein the ablation of the nerve is stacked, comprising a first target ablation site, the first target ablation site being at the inferior end, followed by one or more target ablation sites located superior to the first target ablation site.

13. The method of claim 9, wherein the anesthetic is administered topically, locally or subcutaneously.

14. The method of claim 9, wherein the concentration of anesthetic is about 0.001% volume/volume (v/v) to about 10% v/v.

15. The method of claim 9, wherein the volume of anesthetic administered comprises between about 0.001 ml to about 10 ml.

16. The method of claim 9, wherein the target ablation sites comprise one or more nerves comprising: nerves affecting platysma, angular nerves, temporal nerves, the cervical nerve (VII) or branches of the cervical nerve (VII).
